(19) United States
(12) Patent Application Publication
(10) Pub. No.: US 2022/0243162 A1
(43) Pub. Date: Aug. 4, 2022

(54) BIOREACTOR AND BIOREACTOR SYSTEM FOR CELL AND TISSUE GROWTH

(71) Applicant: CelVivo ApS, Blommenslyst (DK)

(72) Inventors: Krzysztof W. Wrzesinski, Blommenslyst (DK); Stephen John Fey, Blommenslyst (DK)

(21) Appl. No.: 17/623,817

(22) PCT Filed: Jul. 2, 2020
(86) PCT No.: PCT/EP2020/068632

§ 371 Date: Dec. 29, 2021

(30) Foreign Application Priority Data
Jul. 4, 2019 (EP) 19184469.5

(51) Int. Cl. C12M 3/04; C12M 1/12; C12M 1/00; C12M 1/34

(52) U.S. Cl. CPC C12M 27/10; C12M 23/06; C12M 23/34; C12M 23/22; C12M 29/04; C12M 41/44; C12M 29/20

(57) ABSTRACT

The invention relates to a bioreactor (10, 10′, 100) adapted for rotation, the bioreactor comprising: a vessel (12) comprising: a first end (24) and a second end (26) which define a central axis (28) of the vessel (12) extending along a first direction, e.g. a length direction, of the vessel (12) from said first (24) to said second end (26), at least one wall (18, 18′, 20) running along the first direction of the vessel (12), at least one media conduit (22, 22′) defining a volume for receiving fresh or spent media; an inner chamber defined by at least a part of a space confined within said at least one wall (18, 18′, 20) and comprising a fresh media chamber (14) and a spent media chamber (16); a cell culture chamber (30) in fluid communication with said at least one media conduit (22, 22′) and said fresh (14) and/or spent media chamber (16); and a movable wall (38) configured, within said inner chamber, to separate said fresh media chamber (14) from said spent media chamber (16) within said inner chamber.

The present invention concerns a bioreactor device and bioreactor system for the growing of cell cultures and tissues, in particular a bioreactor operated under omnidirectional normogravity conditions, often incorrectly referred to as microgravity conditions, by continuous rotation of a compartment of the bioreactor containing the cell culture or tissue using a clinostat type device. More particularly, the invention concerns a bioreactor in the form of a vessel, where fresh and spent growth media are held within the inner chamber and are separated by a movable wall, such as a piston. The invention also comprises a bioreactor system including the bioreactor and driving unit(s) for displacing the piston and rotating the bioreactor.

Bioreactors for the growing of cell cultures, whether a single or several cell types, or tissues, require normally operation under omnidirectional normogravity conditions i.e. clinostat induced conditions, since this enables the preservation of the differentiated state of many types of cells in the culture or recovery, or (re-)differentiation of in vivo like functionality in cell lines. Such omnidirectional normogravity conditions are induced e.g. by more or less continuous rotation of the compartment containing the cell culture, thereby preventing the cells to adhere to the compartment walls (strictly speaking, the rotation infinitesimally increases the gravitational force (centripetal acceleration)). Suitable rotation promotes the adherence of cells to each other in a fluid environment with a minimum of shear forces acting on the culture. Shear forces can be introduced, if needed, for specific cell/tissue types, by changing the rotation speed of the bioreactor. Thereby cells aggregate into colonies typically named spheroids or organoids (in this disclosure referred to collectively as spheroids). Since pieces of tissue will be affected similarly, they are also included under the generic term spheroids.

Stopping the rotation of the bioreactor device is normally needed in order to change the media or add compounds, e.g. drugs or candidate drugs to the compartment, e.g. an incubation chamber or cell culture chamber. However, this results in the problem that during the stop of the rotation, the spheroids may settle down to the bottom or adhere to the walls of the compartment. This results in turn in a reduction in the availability of gasses, e.g. oxygen, and nutrients to the spheroids thus affecting their quality. Spheroids at the bottom of the sedimented spheroids will suffer more than those at the top. In addition, different users will be more or less skilled in this operation and thus introduce more variability. Thus, stopping the rotation results in changing from active diffusion environment in a rotating bioreactor to static conditions with limited diffusion rates, resulting in suboptimal growth conditions for spheroid population (and the bigger the spheroid size, the more severe effect is observed). In particular, this may cause the spheroids to lose important properties such as having a desirable phenotype. It is thus desirable to be able to reduce the number of times that the rotation has to be stopped to a minimum, or ideally never stop its rotation, so as to keep the spheroids as uniform as possible. When the spheroids are very uniform, it is possible to find a particular rotation speed at which they are in a ‘stationary orbit’ with respect to the media around them and so only tumble very gently in the media solution: this reduces the mechanical shear stress to extremely low levels, i.e. the culture is essentially mechanically ‘stress-free’.

Improved uniformity of the spheroids results in a better metabolic performance which then enables for example a more reliable in vitro predictive toxicological evaluation of candidate drugs prognosis of the cell culture before going into expensive clinical trials or similar, i.e. it results in a more reliable “filter” prior to embarking into clinical trials.

U.S. Pat. No. 9,850,458 addresses this challenge by the provision of a lid as the incubation cavity which is fully accessible. However, while the removal of spheroids according to the teaching of the patent is quicker than the previous art, the uniformity of the spheroids is still somewhat impaired, while at the same time the bioreactor requires handling by the operator for enabling the removal of the spheroids.

EP1966367 A1 discloses a bioreactor for incubation of cell cultures, tissue biopsies, cell clusters, tissue-like structures, ‘proto-tissues’ or similar samples. The bioreactor is adapted for rotation for use in microgravity conditions and equipped with an incubation cavity having a small internal fluid volume, generally less than 1 ml. The small-volume bioreactor permits the use of smaller amounts of reagents which might be of limited supply or might be expensive.

US 2004/0219659 discloses a bioreactor system including components which exert physiologically relevant translational and rotational strains on a growing bioengineered tissue. Thus, physical stress to the cells or tissue is exerted.

US 2013/0230907 discloses a bioreactor designed for generating hydrodynamic pressure, thus subjecting the cell culture to hydrodynamic pressure and thereby exerting physical stress to the cells or tissue.

US 2017/0009207 discloses an apparatus comprising a bioreactor in which the cells therein are exposed to physical stress by means of a mechanical and electrical system. Thus, the apparatus is designed to exert physical stress and/or electrical stimulation to the cells.

US2016/0201037 discloses a bioreactor for growing cells in which the bioreactor device includes a piston. The bioreactor can include an inner body which divides the bioreactor into distinct chambers and facilitates the growth of a multi-tissue sample. The device is designed to grow cells as a layer (or several layers) which are attached to the device. Hence, contact between the device and the cells takes place.

It is therefore an object to provide a bioreactor which enables better uniformity of the spheroids, while at the same time avoiding imposing stress on the cell culture during operation as well as more reproducible handling of the bioreactor, i.e. a bioreactor which is easier to operate.

It is another object to provide a bioreactor that minimises contact between the walls of the bioreactor and the cells or tissue.

It is yet another object to provide a bioreactor which is simple and easy to keep sterile.

It is a further object to provide a bioreactor which enables an even suspension i.e. a substantially stationary orbit which reduces shear forces to a minimum, of the spheroids being produced therein.

It is a further object to provide a bioreactor which enables constant or programmable media supplementation.

It is yet a further object to provide a bioreactor which enables preloading of needed media to bioreactor chambers, further minimising bioreactor handling.

At least one or more of these (and other objects) is/are solved at least to an extent by aspects and embodiments as disclosed herein.

Hence, according to a first aspect, there is provided a bioreactor adapted for rotation, the bioreactor comprising:

a vessel comprising:

a first end and a second end which define a central axis of the vessel extending along a first direction, e.g. a length direction, of the vessel from said first to said second end, at least one wall running along the first direction of the vessel, at least one media conduit defining a volume for receiving fresh or spent media;

an inner chamber defined by at least a part of a space confined within said at least one wall and comprising a fresh media chamber and a spent media chamber;

a cell culture chamber in fluid communication with said at least one media conduit and said fresh and/or spent media chamber; and

a movable wall configured, within said inner chamber, to separate said fresh media chamber from said spent media chamber within said inner chamber.

In some embodiments,
the at least one wall comprises an inner wall and an outer wall, the inner and outer wall defining (or creating) an annular compartment in between said walls,
the at least one media conduit is the annular compartment, and
the inner chamber is defined by at least a part of, e.g. all of, the space confined within said inner wall.

The inner and outer wall, defining or creating the annular compartment, thus form a double-wall vessel in which fresh and spent media (growth media) are retained within the inner chamber of the vessel while being separated by the movable wall.

Preferably, said annular compartment defines a volume for receiving fresh media.

Preferably, said culture chamber is in fluid communication with said annular compartment and said spent media chamber.

In some alternative embodiments,
the at least one wall is a single wall, and
the at least one media conduit is at least one conduit arranged inside or outside the single wall.

Accordingly, in an embodiment of the first aspect, there is provided a bioreactor adapted for rotation, the bioreactor comprising:

a vessel comprising:

a first end and a second end which define a central axis extending of the vessel along a first direction, e.g. a length direction, of the vessel from said first to said second end, at least one wall running along the first direction of the vessel, at least one media conduit defining a volume for receiving fresh media;

an inner chamber defined by at least a part of a space confined within said at least one wall and comprising a fresh media chamber and a spent media chamber;

a cell culture chamber in fluid communication with said at least one media conduit and said spent media chamber; and

a movable wall configured, within said inner chamber, to separate said fresh media chamber from said spent media chamber within said inner chamber.

The first end of the vessel is preferably flat and substantially vertical (or substantially perpendicular to an axis of rotation that e.g. may be substantially horizontal). The second end of the vessel is preferably flat and substantially vertical (or substantially perpendicular to the axis of rotation that e.g. may be substantially horizontal).

Hence, the bioreactor acts as a perfusion bioreactor by which the cell culture in the cell culture chamber is retained and suspended preferably without touching the walls of the vessel, while a source of fresh media providing fresh nutrients is provided and spent media comprising cell waste products is removed. Unlike conventional perfusion bioreactors, the bioreactor as disclosed herein is simple in its construction and does not require tubing to convey fresh media from a fresh media reservoir to the cell culture chamber and to convey spent media from the cell culture chamber to a spent media reservoir, outlet or the like. Furthermore, in contrast to conventional perfusion bioreactors, the bioreactor of the first aspect may also be adapted for rotation to provide a clinostat cell culture environment.

The bioreactor of the first aspect also allows the media to be exchanged in various ways. For example the media could be provided at a constant speed, or it could be provided intermittently e.g. three times a day to mimic breakfast, lunch and dinner, or it could be provided to keep a particular component at a predefined level e.g. by adapting into the bioreactor or bioreactor system a component-sensor which activates media exchange.

Alternatively, the fresh media can be pre-equilibrated before it is filled into the bioreactor.

In some embodiments, gas exchange, e.g. oxygen and carbon dioxide, can occur either through the plastic walls of the vessel, e.g. through polydimethylsiloxane (PDMS) or similar, or through special filters mounted in the walls of the vessel, preferably the walls of the cell culture chamber.

Alternatively, the fresh media can be pre-equilibrated before it is filled into the bioreactor.

The bioreactor is thus simple, integrated, and capable of producing uniform spheroids. The bioreactor is also easier to keep sterile and easier to operate, i.e. by enabling changing or refilling of cell culture only after longer periods, e.g. 14 days or more, rather than typically every 48 hrs or so. This time period can be extended by filling the fresh media chamber with a ‘stock’ solution where one or more of the components that are expected to become exhausted during culture, are present at a higher concentration. For example, if the cultures use glucose, glucose is often provided at a physiological concentration of 1 g/L. If the media in the fresh media chamber contains glucose at 4.5 g/l then the culture can be maintained for 4.5 times the length of time. Media containing both 1 g/L and 4.5 g/L are commercially available from several sources. As an example: for mature C3A spheroids, which are metabolically very active, the media is usually changed each 2 days (other cell types may be different). Considering a cell culture chamber of 10 mL, this means that there would be 7 media changes in 14 days. This would require 70 mL of fresh media. Thus, the media reservoir should be at least 70 mL. If the media contains high levels of glucose (4.5 g/L) then 70 mL would be equivalent to media for 63 days. However, other components in the media may become limiting during this time.

In some embodiments, suitable internal dimensions for a 10 mL cell culture chamber would thus be approximately 3-4 cm in diameter and 1.5-0.8 cm in height and for the media chamber/reservoir would be approximately 3-4 cm in diameter and 10-5 cm in height, without taking into account the volume of connecting elements. These can be scaled correspondingly for vessels of other diameters.

Typical flow rates can be calculated from these numbers. For example, if 70 mL of media has to be changed in 14 days at a constant flow rate, the flow rate would be about 0.2 mL per hour. In other instances where the flow should mimic 3 ‘meals’ a day, each taking 30 minutes, the flow rate would be 3.33 ml per hour during each of the 3 ‘meals’.

In some embodiments of the first aspect, said cell culture chamber is arranged separately at said first end of the vessel and is provided with an inlet orifice for allowing media, e.g. liquid media, from said at least one media conduit, e.g. said annular compartment or said at least one conduit, to enter into the cell culture chamber, and an outlet orifice or valve for allowing media, e.g. liquid media, from said culture chamber to enter into said spent media chamber. The movable wall enables that fresh media flows through the at least one media conduit to the cell culture chamber, while the orifice and particularly a valve (if present) prevents the mixing of the two liquids, i.e. the cell culture and the spent media.

The bioreactor of the first aspect results also in that the use of filters to separate the cell culture chamber from either the fresh media chamber, media conduit and spent media chamber becomes optional. Rotation of the bioreactor will tend to cause the spheroids to move away from the central axis, i.e. away from the outlet port of the cell culture chamber. The influx of media, at even a slow rate, will prevent spheroids from entering the media conduit. Thus the use of a membrane in the bioreactor to prevent spheroids from leaving the cell culture chamber is avoided, thus resulting in a much simpler an inexpensive construction.

Yet, in a particular embodiment of the bioreactor according to the first aspect, the bioreactor includes a membrane for preventing at least part of the cell culture in the cell culture chamber from exiting said cell culture chamber. Preferably, the membrane (one or more) is (are) arranged over or at least in connection with the inlet and/or outlet orifice(s) of the cell culture chamber. This enables holding components, cells or spheroids in the cell culture chamber and also prevents components or cells, e.g. microorganisms, from entering or exiting the cell chamber. In some embodiments, such a membrane is e.g. located at an outlet orifice or valve (see more in the following) for allowing media (liquid media) from said culture chamber to enter into said spent media chamber. Alternatively, or in addition, such a membrane is e.g. located at an inlet orifice or valve (of the cell culture chamber) for allowing media (liquid media) from the annular compartment or the media conduit to enter into the cell culture chamber. As yet another alternative or in addition, such a membrane is e.g. located at an (e.g. circumferential) access port or the like providing access to the cell culture of the cell culture chamber. In some embodiments, a single such membrane, e.g. a membrane disk or the like, may cover two or even all three of the above locations, in particular the single membrane may cover the outlet orifice or valve for allowing media (liquid media) from said culture chamber to enter into said spent media chamber and the inlet orifice or valve (of the cell culture chamber) for allowing media (liquid media) from the annular compartment or the media conduit to enter into the cell culture chamber.

Hence, there may be situations where such a membrane is advantageous. These situations include when microorganisms are cultured, or co-cultured with spheroids which are too small to be significantly influenced either by gravity or by media flow, or they may be motile, or when the user desires to retain compounds preferentially inside or outside of the cell growth chamber.

In another embodiment of the first aspect, said at least one media conduit, e.g. said annular compartment or said at least one conduit, is provided with an inlet orifice for allowing media (i.e. liquid, or liquid media) from said fresh media chamber to enter into the at least one media conduit, and an outlet orifice for allowing media from the at least one media conduit to enter into the cell culture chamber, and wherein said outlet orifice corresponds to said inlet orifice of the cell culture chamber. This facilitates the flow of fresh media into the cell culture chamber. Accordingly, the fresh media chamber is provided with an outlet orifice for allowing media e.g. liquid media from said fresh media chamber to enter into the at least one media conduit, where this outlet orifice of the at least one media conduit corresponds to the above inlet orifice of said at least one media conduit.

In another embodiment of the first aspect, at least part of said at least one wall is detachable, preferably as a removable end of the at least one wall located at said first end of the vessel, for providing access to the cell culture chamber. This enables a much simpler construction of the bioreactor and easier maintenance due to better accessibility to the bioreactor's internal parts, in particular to the cell culture chamber and the cells or spheroids therein. By the term “detachable” is meant that it can be reversibly opened or removed. In a particular embodiment, a flat part of the removable end of the at least one wall located at said first end of the vessel includes at least one access port and optionally a sensor, said sensor preferably being mounted in said access port. In another particular embodiment the sensor is mounted in its own location in the chamber wall.

In another embodiment of the first aspect, at least part of said at least one wall is transparent glass or plastic configured to permit observation of the media, cells and spheroids contained therein, e.g. the removable end for such embodiments. This enables that the cell culture be monitored and assessed from the outside. The glass or plastic, apart from being transparent, is preferably also biologically and chemically inert with the media and cells. Furthermore, the plastic should preferably have low affinity to bind, adsorb, or absorb any compounds in the media including for example bioactives or candidate drugs. As suitable plastics, polystyrene, polypropylene and polyethylene may be used.

In some embodiments, the vessel including chambers may be made of glass-clear plastic, while all other components are made of polypropylene.

In other embodiments of the first aspect (having an inner wall and an outer wall defining an annular compartment in between said walls), said inner wall has a plurality of raised ridges extending along the first/length direction of the vessel, and extending vertically outwards, i.e. radially-perpendicular to said first/length direction, until contacting said outer wall, and wherein the space between said raised ridges define one or more sub-compartments within said annular compartment. The raised ridges are preferably arranged in parallel. The raised ridges enable the creation of a better flow of the media in the annular compartment, as this is divided in a number of sub-compartments which better approach a laminar flow condition, thus avoiding back-mixing and turbulent flow that may damage the media flowing therein. The provision of the raised ridges as recited above enables also a simpler construction and thus the manufacturing of the bioreactor in an inexpensive way.

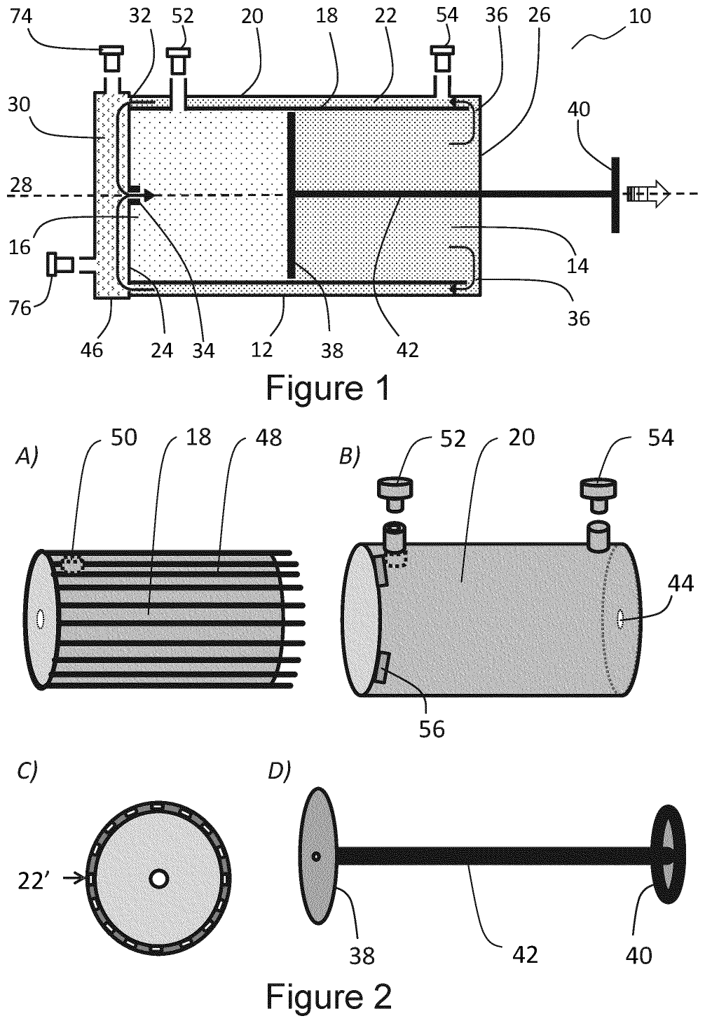

In another embodiment of the first aspect, the vessel has a cylindrical or generally cylindrical shape and is adapted for rotation around a rotational, e.g. horizontal, axis by one or more associated rotation elements (e.g. a drive unit), said rotational axis being said central axis running along the first/length direction of the vessel. The rotation enables the provision of omnidirectional normogravity conditions, meaning that spheroids being formed in the cell culture chamber readily may be suspended in a stable orbit without touching the walls of the vessel. The horizontal rotational axis may be as defined by the central axis line denoted 28 in FIG. 1.

In another embodiment of the first aspect, said movable wall is connected to a displacement element, for displacing the movable wall axially along the first/length direction of the vessel, preferably in a direction away from said cell culture chamber. In circumstances where the flow needs to be reversed, the movable wall will be displaced axially along the first/length direction of the vessel in a direction towards said cell culture chamber. In a particular embodiment, said displacement element is a piston, said movable wall being connected to said piston through a piston shaft which e.g. or preferably is coincident with said central axis running along the first/length direction of the vessel. In another particular embodiment, a conduit is provided which runs from the cell culture chamber through a centre of said piston shaft to at least the outside of the vessel. In yet another particular embodiment, the piston shaft is a conduit such as a tube which runs from the cell culture chamber to at least the outside of the vessel. This enables easier and straightforward sampling and monitoring of the cells in the culture chamber. Hence, it permits on-line sampling of media or cells in this chamber without resorting to having to stop the rotation of the bioreactor. Furthermore, from the outside it is possible not only to collect spent media, but also to introduce fresh media, compounds e.g. bioactive molecules, drugs or candidate drugs, or other solutions.

In another embodiment of the first aspect, the vessel is rotated at a speed of 0.1-200 rpm. The optimal speed of rotation of the vessel depends on many factors including the age of the spheroids since as they get older, they get larger and require a higher rpm to reach the ‘stationary orbit conditions’, the temperature and the viscosity of the media used. Fine regulation of the rpm, e.g. to 0.1 rpm, may be necessary to reach the ‘stationary orbit condition’. Rotation should preferably be essentially free of vibration, wow, and flutter.

In another embodiment of the first aspect, the vessel or part of the vessel is constructed of a gas permeable plastic, or the vessel includes a gas permeable membrane for the exchange of gasses such as oxygen and carbon dioxide. In a particular embodiment, said gas permeable membrane is arranged along said first or second ends of the vessel. In another particular embodiment, said gas permeable membrane is arranged along the circumferential part i.e. the perimeter, of the cell culture chamber, preferably along the circumferential part of said removable end of the outer wall located at said first end of the vessel. Preferably, said gas permeable membrane is a semipermeable membrane.

While the vessel is preferably cylindrically shaped and thereby its cross-sectional perimeter is circular as so is the cell culture chamber arranged therein, the cross-sectional perimeter of the vessel can be other than circular, such as a polygon.

In another embodiment of the first aspect, a humidification system is provided between the culture chamber and the external atmosphere, i.e. the surrounding atmosphere outside the vessel, said humidification system comprising a liquid reservoir preferably containing sterile water, an evaporation chamber such as an evaporation labyrinth and a filter such as a gas permeable membrane, particularly a semipermeable membrane. This enables facilitating the exchange of gasses into the cell culture chamber while simultaneously avoiding the loss of water from the culture chamber. Preferably, said filter is said gas permeable membrane arranged along the circumferential part i.e. perimeter of the cell culture chamber. In an embodiment, said evaporation chamber e.g. evaporation labyrinth and said liquid reservoir are arranged along the circumferential part of the vessel, in the same manner as the gas-permeable membrane.

In yet another embodiment, the humidification system further comprises an additional filter being arranged in fluid communication with said liquid reservoir and said evaporation chamber, thereby allowing evaporation from the liquid reservoir into the evaporation chamber e.g. evaporation labyrinth. The additional filter is preferably arranged along the circumferential part of the vessel and in between said liquid reservoir and said evaporation chamber. The additional filter is preferably highly permeable to allow evaporation from the liquid reservoir into the evaporation chamber. The additional filter may be a wick or a gas-permeable filter. The latter is most suitable for an embodiment in which the additional filter is arranged along the circumferential part of the vessel. Such gas-permeable filter will then have different properties to the above semipermeable membrane arranged along the circumferential part of the cell chamber.

In a particular embodiment, the humidification system further comprises at least one opening port connecting the evaporation chamber e.g. evaporation labyrinth with the external atmosphere.

In a particular embodiment, a port is provided for allowing air to be sucked into the spent media chamber. This may be necessary, in the event that a negative pressure develops in the vessel.

In another embodiment of the first aspect, the cell culture chamber includes at least one access port and optionally a sensor, said sensor preferably being removably mounted in said access port. By being mounted is meant that the sensor is integrated in the access port. This represents an elegant and simple way to continuously measuring the state or conditions of the cell culture. For instance, the sensor mounted in the access port may be adapted to measure the glucose level of the cell culture and if the glucose level is low, activate the piston via a control mechanism so it is pulled out and thus enable the replenishment of the cell culture chamber with fresh media, including fresh glucose.

In another embodiment of the first aspect, at least one sensor is mounted on the part of the at least one wall, e.g. an inner wall, of the vessel which is in direct contact with the cell culture chamber.

In another embodiment of the first aspect, said at least one wall, e.g. an outer wall, includes an access port to said fresh media chamber. This enables the provision of fresh media in the bioreactor, which is particularly relevant at the beginning of use. This access port is directly connected to the fresh media chamber and is preferably located at the periphery of the at least one wall, e.g. outer wall, at the second end of the vessel, i.e. farthest away from the cell culture chamber.

In another embodiment of the first aspect, said at least one wall, e.g. outer wall, includes a port for introducing or removing air, liquids, cells, cell aggregates, or biologically active molecules, e.g. drugs. This access port may be the same access port as the above access port to said fresh media chamber. Preferably, the access port is arranged on the circumferential side of the vessel or on the removable part of the at least one wall, e.g. outer wall, of the vessel.

Any of the access ports or ports has preferably a cup around it to facilitate keeping the region around the access port clean, dry and sterile and facilitate the removal of air bubbles.

In another embodiment of the first aspect, an additional cell culture chamber is adapted in series connection with said cell culture chamber, said additional cell culture chamber having a conduit to transfer said fresh media and having an orifice for allowing the media to flow from the cell culture chamber to said additional cell culture chamber. Hence, an extra, preferably petri-dish shaped cell culture is interposed between the removable end of the vessel (i.e. vessel according to the first aspect) and the rest of the bioreactor, such an additional cell culture chamber having conduits to transfer the fresh media from the rest of the bioreactor to the removable end of the vessel and having a hole or orifice to allow the media to flow between the cell culture chamber and the additional cell culture chamber. In a particular embodiment, one or more additional cell culture chambers are assembled on or in the vessel by inserting extra additional cell culture chambers, preferably essentially petri-dish shaped cell culture chambers. In another particular embodiment, at least one sensor is mounted on or in the one or more additional cell culture chambers in such a way that the contents of each chamber can be monitored independently.

For a connected series of cell culture chambers it is noted, that the cell culture chamber at one or both ends (i.e. the first and/or the last) may comprise a double vent (as disclosed herein) located on the front or back rather than a top-side port, while any cell culture chambers in-between should comprise a top-side port or any other suitable port to enable exchange with outside or ambient air or gas of the bioreactor for a gas exchanger and, if present, a humidifier.

In a second aspect, a bioreactor (where the at least one wall comprises an inner wall and an outer wall defining an annular compartment in between said walls) is provided in which the cell culture chamber is in the annular portion (i.e. the annular compartment) of the vessel. Accordingly, there is provided a bioreactor adapted for rotation, the bioreactor comprising:

a vessel including:

a first end and a second end which define a central axis of the vessel extending along a first direction, e.g. a length direction, of the vessel from said first to said second end,

at least one wall running along the first direction of the vessel;

at least one media conduit defining a volume for receiving fresh media;

an inner chamber defined by at least a part of a space confined within said at least one wall and comprising a fresh media chamber and a spent media chamber;

said at least one media conduit being or comprising a cell culture chamber in fluid communication with said spent media chamber and said fresh media chamber;

a movable wall configured, within said inner chamber, to separate said fresh media chamber from said spent media chamber within said inner chamber.

In some embodiments
the at least one wall comprises an inner wall and an outer wall, the inner and outer wall defining (or creating) an annular compartment in between said walls,
the at least one media conduit is the annular compartment, and
the inner chamber is defined by at least a part of, e.g. all of, the space confined within said inner wall.

In some embodiments,
the at least one wall is a single wall, and
the at least one media conduit is at least one conduit arranged inside or outside the single wall.

Hence, the cell culture chamber is an annulus (or comprised in the one or more media conduits), i.e. the cell culture chamber occupies the annular compartment or sub-compartments (or the one or more media conduits). This enables a more compact construction of the bioreactor and is well suited for small cell culture chamber volumes. The average radial distance is greater making it easier to find the ‘stationary orbit’ rotation speed.

In a third aspect, a bioreactor system is provided for growing a cell culture or tissue, the system comprising:

a bioreactor adapted for rotation, said bioreactor comprising a vessel comprising:

a first end and a second end which define a central axis of the vessel extending along a first direction, e.g. a length direction, of the vessel from said first to said second end, at least one wall running along the first direction of the vessel, at least one media conduit defining a volume for receiving fresh or spent media, preferably fresh media;

an inner chamber defined by at least a part of a space confined within said at least one wall and comprising a fresh media chamber and a spent media chamber;

a cell culture chamber in fluid communication with said annular compartment and said fresh or spent media chamber, preferably said media chamber; and

a movable wall configured, within said inner chamber, to separate said fresh media chamber from said spent media chamber within said inner chamber;

wherein said movable wall is connected to a displacement element in the form of a piston, for displacing the movable wall axially along the first direction of the vessel;

the bioreactor system further comprising:
retaining rollers adapted for supporting and enabling rotation of the bioreactor;
a drive element such as drive wheel for rotating the bioreactor;
a retaining block for supporting the piston, said piston being connected to said movable wall via a piston shaft;
a driving unit for moving said retaining block and thereby displacing the piston.

Preferably, the drive element and drive unit for rotating the bioreactor and for moving said retaining block is a syringe pump.

In some embodiments,
the at least one wall comprises an inner wall and an outer wall, the inner and outer wall defining (or creating) an annular compartment in between said walls,
the at least one media conduit is the annular compartment, and
the inner chamber is defined by at least a part of, e.g. all of, the space confined within said inner wall.

In some embodiments,
the at least one wall is a single wall, and
the at least one media conduit is at least one conduit arranged inside or outside the single wall.

In a fourth aspect, a bioreactor system is provided for growing a cell culture or tissue, where the cell culture chamber of the bioreactor is in an annulus, i.e. the annular compartment.

Accordingly, there is provided a bioreactor system for growing a cell culture or tissue, the system comprising:

a bioreactor adapted for rotation, said bioreactor comprising a vessel including:

a first end and a second end which define a central axis extending along a first direction,

e.g. a length direction, of the vessel from said first to said second end, an outer wall and inner wall running along the first direction of the vessel for creating an annular compartment in between said walls;

an inner chamber defined by the space confined within said inner wall and comprising a fresh media chamber and a spent media chamber;

said annular compartment being a cell culture chamber in fluid communication with said spent media chamber and said fresh media chamber;

a movable wall configured, within said inner chamber, to separate said fresh media chamber from said spent media chamber within said inner chamber;

wherein said movable wall is connected to a displacement element in the form of a piston, for displacing the movable wall axially along the first direction of the vessel;

the bioreactor system further comprising:

retaining rollers adapted to support and enable rotation of the bioreactor;

a drive element such as drive wheel for rotating the bioreactor;

retaining block for supporting the piston, said piston being connected to said movable wall via a piston shaft; and

a drive unit for moving said retaining block and thereby displacing the piston.

According to a fifth aspect is provided a bioreactor for the growing of cell cultures and tissues, the bioreactor comprising
a cell culture chamber configured to contain a cell culture media,
a circumferential gas exchanger arranged circumferentially about or along at least a part of the cell culture chamber or about a central or lengthwise axis of the bioreactor, e.g. or preferably about a predetermined rotational axis of the bioreactor, wherein the circumferential gas exchanger comprises a cavity comprising a volume connecting a gas exchange interface of the cell culture chamber with ambient air or gas of the bioreactor.

In this way, the gas exchanger is arranged off centre (but typically still about the central and/or rotational axis) and away from the lengthwise axis (typically extending between the first and the second ends and being substantially parallel to the rotational axis), i.e. the gas exchanger is not ‘stacked’ next to the enclosure or any other component in a lengthwise direction. By having a circumferential gas exchanger, the lengthwise extent of the cell culture chamber device is also greatly reduced reducing the lengthwise ‘footprint’/form-factor which may be beneficial for design considerations.

It is noted, that even though the provision of such a circumferential gas exchanger functions particularly well according to the first aspect, it may be used independently thereof.

In some embodiments, the gas exchange interface is a circumferential gas permeable membrane, e.g. a semipermeable membrane, either porous or non-porous, configured to exchange gases, such as oxygen and carbon dioxide, with the content of the cell culture chamber, where the circumferential gas permeable membrane is arranged circumferentially along a circumferential part of the cell culture chamber.

In some embodiments, the circumferential gas permeable membrane is a connecting wall connecting a first end and a second end wherein the first end, the second end, and the connecting wall at least in part defines the cell culture chamber.

In some embodiments, the gas exchange interface or the circumferential gas permeable membrane is supported by at least one support structure, e.g. a grid like support structure, comprising a number of openings configured to connect the gas exchange interface or the circumferential gas permeable membrane with air or gas of the cavity of the circumferential gas exchanger.

In some embodiments, the circumferential gas exchanger is connected with the ambient air or gas of the bioreactor via at least one gas or air inlet and/or outlet.

In some embodiments, the bioreactor is configured for rotation about a predetermined rotational axis and wherein at least one of the at least one gas or air inlet and/or outlet is a double vent or port configured to, e.g. or preferably simultaneously, draw in ambient air or gas into the cavity of the circumferential gas exchanger and expel air or gas out of the cavity of the circumferential gas exchanger in response to the bioreactor being rotated about the predetermined rotational axis thereby creating an air flow.

In some embodiments, the bioreactor further comprises
a circumferential humidifier, wherein the circumferential humidifier
   is arranged circumferentially about at least a part of the cell culture chamber or about a central or lengthwise axis of the bioreactor, e.g. or preferably about a predetermined rotational axis of the bioreactor, and
   comprises or is connected to one or more liquid or moisturising reservoirs or elements configured to humidify or moisturise air or gas in at least a part of a cavity of the gas exchanger.

In some embodiments, the one or more liquid or moisturising reservoirs or elements is/are configured to humidify or moisturise air or gas in vicinity of or being adjacent to at least a part of a gas exchange interface or a gas permeable membrane.

According to a sixth aspect is provided a bioreactor adapted for rotation, the bioreactor comprising:

a vessel including:
a first end and a second end which define a central axis of the vessel extending along a first direction, e.g. a length direction, of the vessel from said first to said second end, at least one wall running along the first direction of the vessel, at least one media conduit defining a volume for receiving fresh or spent media;
an inner chamber defined by at least a part of a space confined within said at least one wall and comprising a fresh media chamber and a spent media chamber;
a cell culture chamber in fluid communication with said at least one media conduit and said fresh and/or spent media chamber; and
a movable wall configured, within said inner chamber, to separate said fresh media chamber from said spent media chamber within said inner chamber,

wherein the bioreactor further comprises a humidification system comprising
one or more liquid or moisturising reservoirs or elements,
an evaporation chamber, e.g. an evaporation labyrinth and a filter, the filter forming one of the walls of the cell culture chamber, and
and a liquid or moisturizing transport element, e.g. a wick or the like, configured to transport liquid or moisture from the one or more liquid or moisturising reservoirs or elements to the evaporation chamber.

Humidified atmosphere will according be in direct contact with the filter, such as semipermeable membrane, forming one of the walls of the culture chamber. Accordingly, gasses can be relatively freely exchanged between the culture chamber and the atmosphere around the equipment without a net evaporation of liquids from the culture chamber.

In some embodiments, the humidification system is e.g. provided with a port for filling and refilling the liquid reservoir (e.g. with a liquid, typically sterile water) and at least one open port connecting the evaporation chamber or the evaporation labyrinth with the external atmosphere. In some embodiments, the humidification system is arranged between the cell culture chamber and the spent media chamber. An outlet orifice or valve for allowing media (liquid media) from said culture chamber to enter into said spent media chamber may pass, e.g. centrally, through (without mixing) the liquid or moisturising reservoirs or elements to the spent media chamber. As an alternative to using a liquid, the humidifier may e.g. comprise one or more of a water or solute-containing material such as a gel, sponge, a particulate material (e.g. water-beads, aqua-beads, slush powder, or water gel powder, etc.).

Any of the embodiments of the first aspect may be used together with the second or third or fourth or fifth or sixth aspect of the invention. Any of the embodiments of the sixth aspect may be used together with any one or more of the other aspects.

Aspects of the invention is illustrated by the accompanying drawings, where:

FIG. 1 is an embodiment of the bioreactor according to the first aspect, where the cell culture chamber is at the first flat end of the vessel;

FIG. 2 shows internal parts of the vessel according to the first aspect to illustrate a simple way to construct the bioreactor;

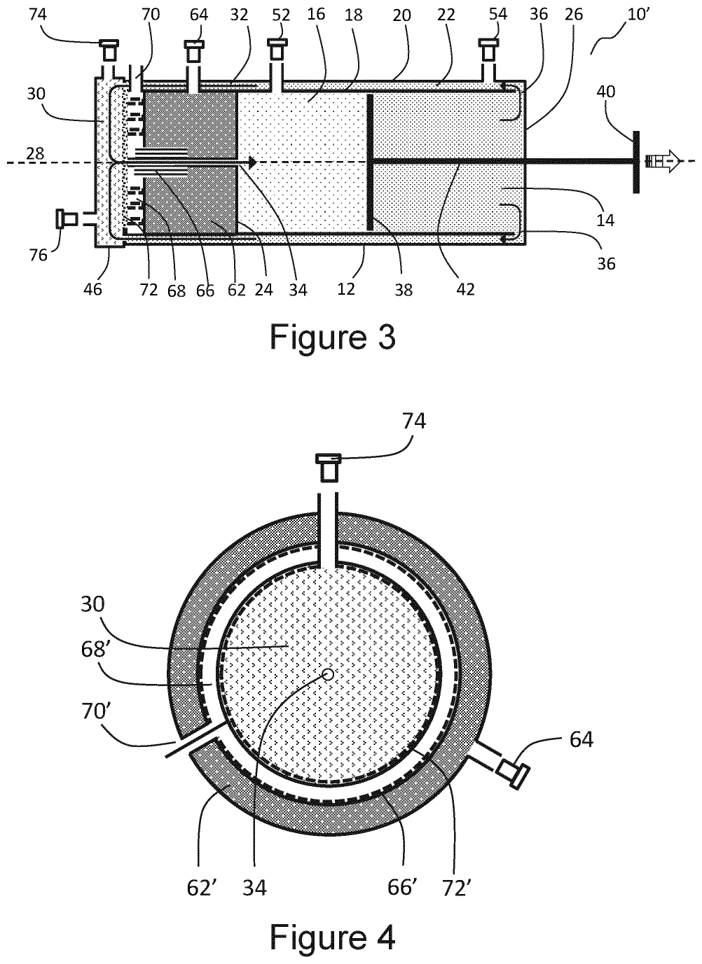

FIG. 3 is an embodiment of the bioreactor according to the first aspect, where a humidification system including a filter is provided between the cell culture chamber and the spent media chamber;

FIG. 4 is an embodiment of the bioreactor according to the first aspect, where a humidification system including a filter is provided along the circumference of the cell culture chamber, and where the humidification system is provided between the culture chamber and the external atmosphere;

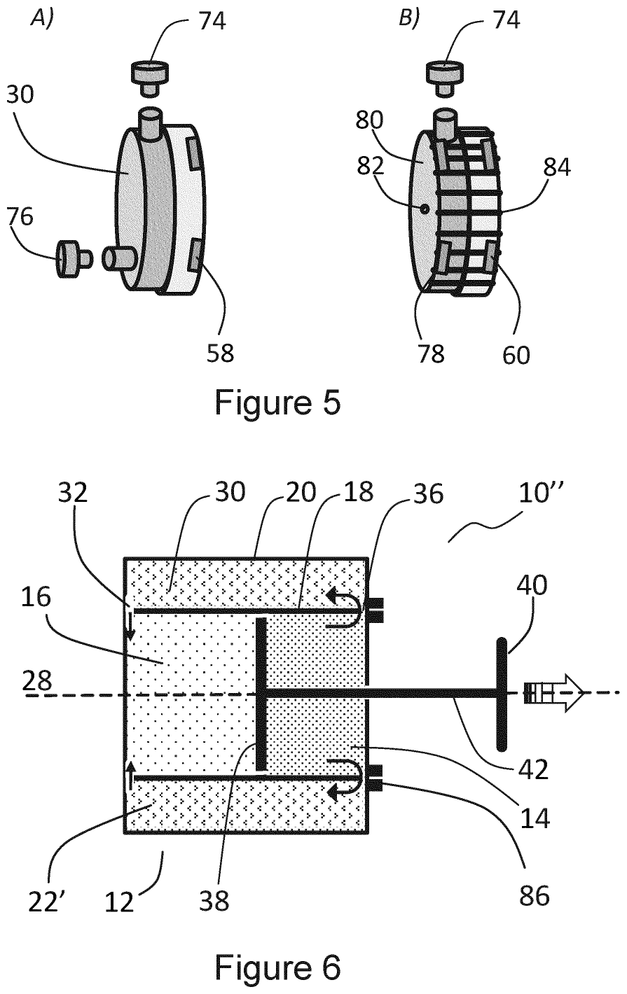

FIG. 5 shows embodiments related to the cell culture chamber 30 and additional cell culture chamber 80;

FIG. 6 is an embodiment of the bioreactor according to the second aspect, where the cell culture chamber is an annulus of the vessel;

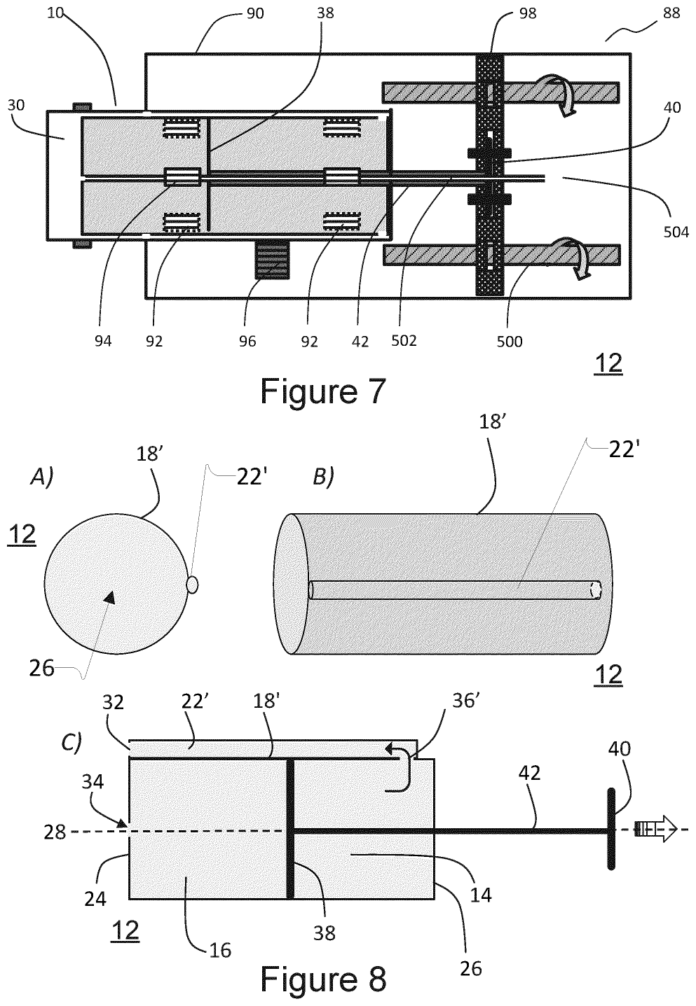

FIG. 7 shows a bioreactor system according to the third aspect, which includes the bioreactor itself, driving element and drive unit for rotating the bioreactor and for displacing the piston;

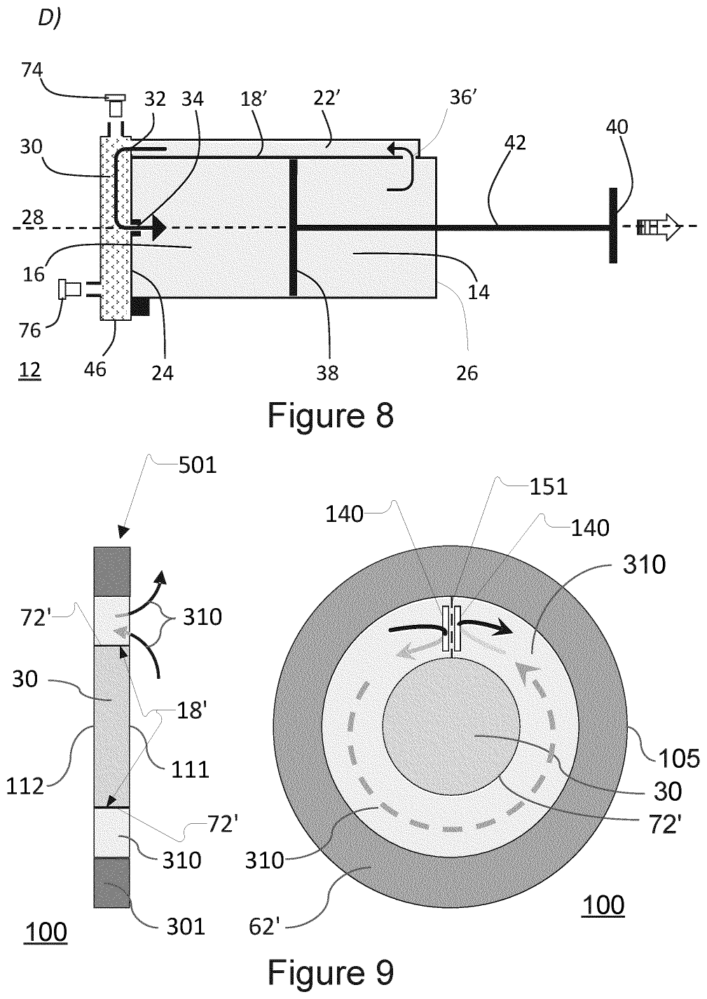

FIG. 8 schematically illustrates an alternative embodiment of a bioreactor according to the first aspect;

FIG. 9 schematically illustrates a front view and a cross sectional side view of embodiments of a bioreactor according to some embodiments and as disclosed herein comprising a circumferential gas exchanger and a circumferential humidifier;

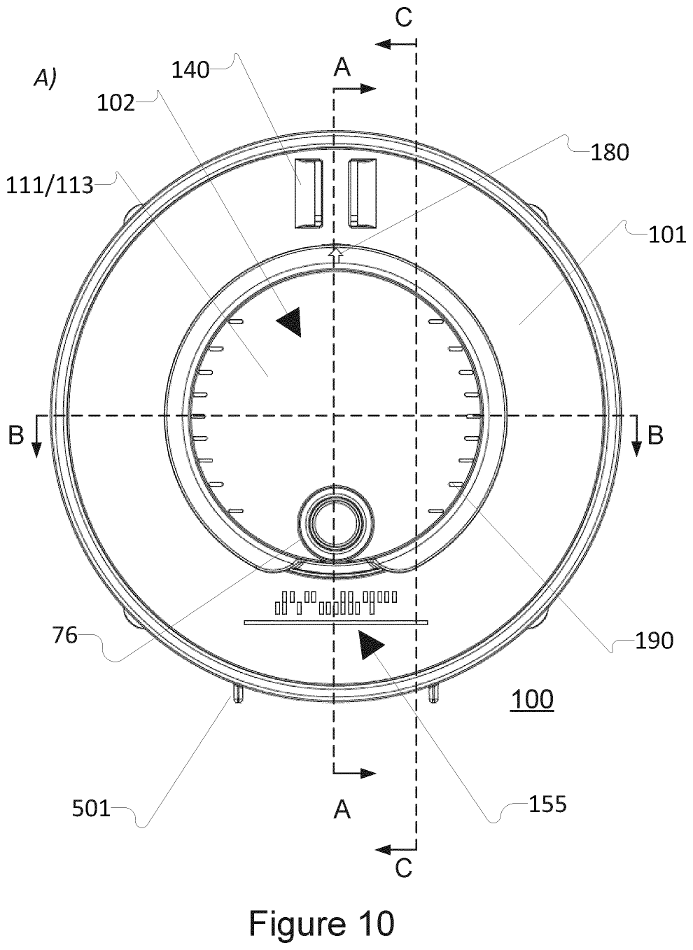

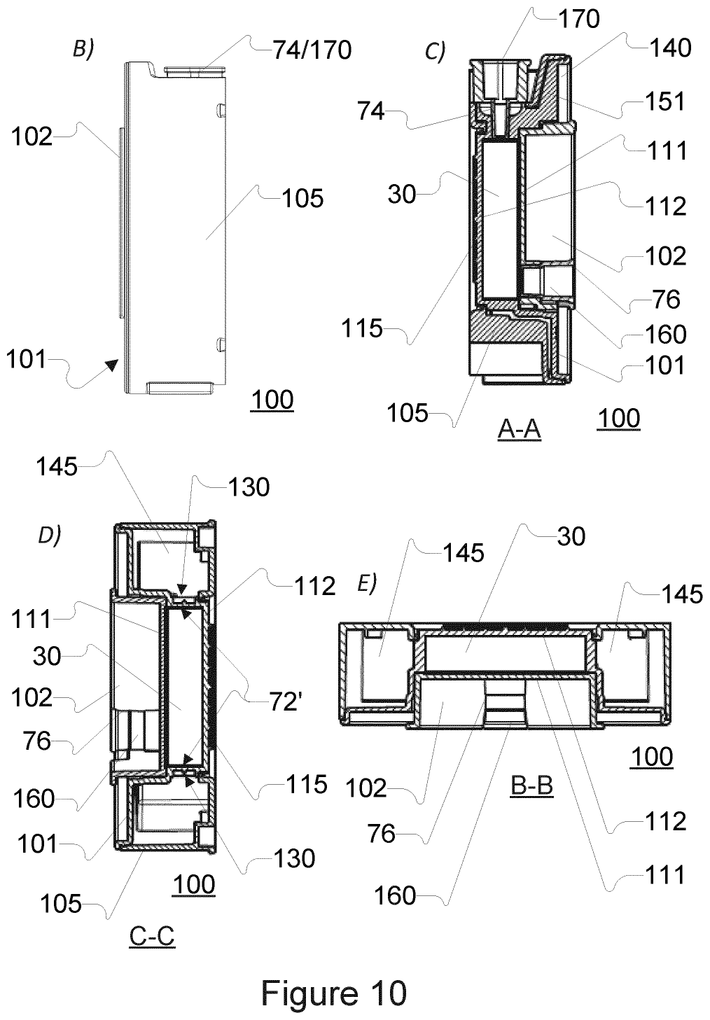

FIGS. 10A-10E respectively schematically illustrates a front, a first (‘right’) side view, a first cross sectional view (AA), a second cross sectional view (CC), and a third cross sectional view (BB) of one exemplary embodiment of a cell culture chamber device as disclosed herein; and

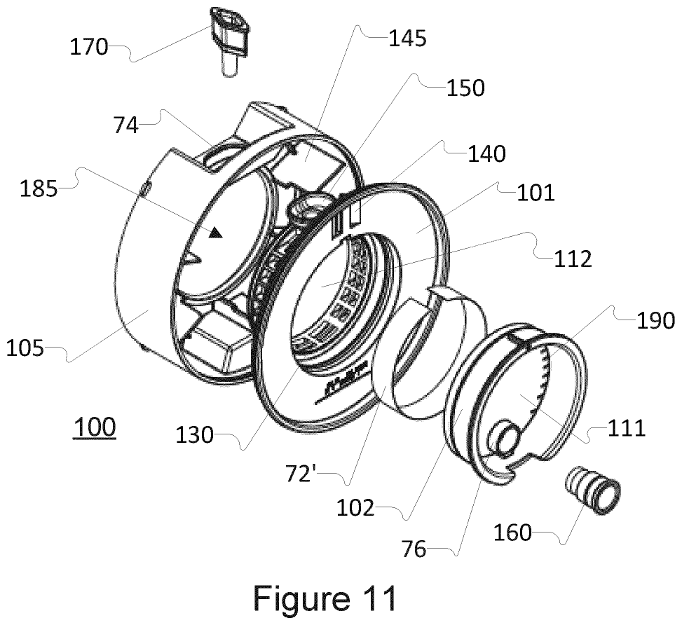

FIG. 11 schematically illustrates a perspective exploded view of the exemplary embodiment of a cell culture chamber device of FIGS. 10A-10E.

With reference to FIG. 1, the bioreactor 10 comprises a vessel 12 which includes an inner chamber comprising a fresh media chamber 14 and spent media chamber 16. In this and corresponding embodiments, the vessel 12 comprises also an inner wall 18 and outer wall 20, both walls running along a first direction (in the shown example being a length direction) of the vessel 12 for creating an annular compartment 22 in between these walls, and thus forming a double wall vessel. The annular compartment 22 may be one single compartment in the form of a single conduit i.e. a single media conduit, or a number of sub-compartments or a number of conduits e.g. as shown in FIG. 2c. The vessel 12 includes a first end 24, preferably a first flat end, and a second end 26, preferably second flat end, at the opposite side of the vessel 12. The first 24 and second 26 ends define a central axis 28, as shown by the horizontal stippled line, which extends along the length direction of the vessel from said first end 24 to said second end 26. The vessel 12 comprises also a cell culture chamber 30 arranged at said first end 24 of the vessel, as well as a movable wall 38 adapted within the inner chamber for separating the fresh media chamber 14 from said spent media chamber 16, while keeping at least a part of said fresh and spent media (as shown by dotted areas) within the inner chamber. The cell culture chamber 30 has an inlet orifice 32 for allowing media (liquid media) from the annular compartment 22 to enter into the cell culture chamber 30, as well as an outlet orifice or valve 34 for allowing media (liquid media) from said culture chamber 30 to enter into said spent media chamber 16. Likewise, the annular compartment 22 has an inlet orifice 36 for allowing media from the fresh media chamber 14 to enter into the annular compartment 22, and an outlet orifice 32 for allowing liquid/media from the annular compartment 22 to enter into the cell culture chamber 30. The outlet orifice of the annular compartment corresponds to or is coincident with the above-mentioned inlet orifice 32 of the cell culture chamber 30. The movable wall 38 is connected to piston 40 via a piston shaft 42. When the piston moves in the right direction (in the orientation of the Figure), as shown by the arrow, the movable wall 38 is displaced and thereby fresh media from the fresh media chamber 14 passes through the annular compartment 22 into the cell culture chamber 30. The vessel 12 comprises also a removable end 46 (comprising the cell culture chamber 30) of the outer wall 20, which is located at said first end 24 of the vessel. The vessel may e.g. also comprises (as shown) a circumferential access port 74 or the like providing access to the cell culture of the cell culture chamber 30.

FIG. 2 shows the internal parts of the bioreactor and illustrates the simple design and construction of the bioreactor by quickly assembling such parts.

FIG. 2a shows the inner cylinder of an embodiment of the vessel, comprising inner wall 18 and a plurality of raised ridges 48 extending along a first direction (in the shown example being a length direction) of the inner wall 18 and thereby vessel 12, and each also extending vertically outwards, i.e. perpendicularly to said length direction, in a radial direction, until contacting said outer wall 20. The space between said raised ridges 48 defines one or more sub-compartments 22′ within said annular compartment 22, as shown in the side view presented in FIG. 2c. FIG. 2a shows also hole (orifice) 50 through which access port 52 (FIG. 2b) reaches spent media chamber 16.

FIG. 2b shows the outer cylinder of the vessel, comprising outer wall 20. The outer wall 20 is detachable and comprises access port 52 which is adapted to cooperate with hole 50 of the inner wall 18 (FIG. 2a). The access port 52 serves also as a pressure equalization port, e.g. for relieving pressure differences. The outer wall 20 includes access port 54 (and stopper) for allowing the introduction of fresh media into fresh media chamber 14. A locking flange 56 is also provided, which engages with corresponding flange 58 or 60 (FIG. 5a or b) of a cell culture chamber 30 or an additional cell culture chamber 80.

FIG. 2c is a cross section when the inner cylinder is fitted into the outer cylinder. The raised ridges 48 arranged in parallel create a number of annular sub-compartments or conduits 22′.

FIG. 2d shows an internal part of the vessel in the form of movable wall 38 which is connected to piston 40 via piston shaft 42. When the bioreactor is assembled, the piston shaft 42 passes through orifice 44 in the flat end 26 of the outer wall.

FIG. 3 shows an embodiment of the bioreactor according to the first aspect as disclosed herein, where a humidification system is inserted between the cell culture chamber and the spent media chamber. The humidification system, i.e. humidifier, comprises a liquid reservoir 62, evaporation chamber such as an evaporation labyrinth 68 and filter 72. A liquid, typically sterile water, would be filled into the liquid reservoir 62 via port 64. This liquid would be conveyed via wick 66 to the evaporation labyrinth 68 where it would humidify the atmosphere of the evaporation chamber. The evaporation labyrinth 68 has at least one open port 70 to the atmosphere surrounding the equipment by which gasses, e.g. oxygen and carbon dioxide, can be exchanged. The humidified atmosphere is in direct contact with a filter such as semipermeable membrane 72, which forms one of the walls of the culture chamber 30. By this means, gasses can be relatively freely exchanged between the culture chamber 30 and the atmosphere around the equipment without a net evaporation of liquids from the culture chamber 30. In practice water is lost from the liquid reservoir 62 at a very approximate rate of 1-2 mL per week. Consequently, the reservoir 62 should have a volume of 10-20 mL for a 10 mL bioreactor as described here. The filter 72, evaporation labyrinth 68, and liquid reservoir 62 have a conduit which connects the cell culture chamber with the spent media chamber.

FIG. 4 shows an embodiment of the bioreactor according to the first aspect, where the humidification system includes a filter in the form of semipermeable membrane 72′, which is arranged circumferentially around the cell culture chamber 30, i.e. along the circumferential part of the cell culture chamber. The humidification system includes said filter 72′, liquid reservoir 62′ containing sterile water, additional filter such as wick or gas permeable filter 66′, evaporation chamber in the form of evaporation labyrinth 68′. The additional filter 66′ would be highly permeable to allow evaporation from the liquid reservoir 62′ into the evaporation labyrinth 68′. Hence, in this embodiment, it will be simpler to replace the wick 66 of the embodiment of FIG. 3 with a gas-permeable filter 66′, which will then have different properties to the semipermeable membrane 72′. The humidification system is also provided with port 64 for refilling of liquid reservoir 62′ and at least one open port 70′ connecting the evaporation labyrinth 68′ with the external atmosphere. For access to the cell culture chamber 30 of the bioreactor, port 74 is arranged as already described in e.g. FIG. 1.

The embodiment of the bioreactor shown in FIG. 4 is preferential to that shown in FIG. 1 because the presence of the humidification system will greatly enhance the gas exchange between the cell culture chamber 30 (or additional culture chamber(s) 80) and the surrounding atmosphere and will reduce or eliminate water loss from said cell culture chambers 30 or 80. The difference is so significant that the bioreactor will normally be able to be used in an incubator without additional humidification. This is a great advantage because it will reduce the risk of infection in the incubator.

Accordingly, a circumferential gas exchanger and a circumferential humidification system is readily provided. It is noted, that a circumferential gas exchanger (and a circumferential humidification system) may be provided independent of other features of the first aspect. Please also refer to FIG. 9 and related description for an alternative embodiment of a circumferential gas exchanger and a circumferential humidification system according to the fifth aspect and as disclosed herein.

The embodiment of the bioreactor shown in FIG. 4 is also preferential to that shown in FIG. 3 for at least three reasons. The first is that it will be easier to regulate the gas exchange by having a stopper or sliding cover which can partially or completely close port 70′ so that it is possible to regulate gas exchange of an individual bioreactor, rather than having to regulate gas atmosphere of the whole incubator. The second reason is that the bioreactor illustrated is more compact along the axis 28 and finally is less expensive to manufacture. In addition, a circumferential gas exchanger (and circumferential humidifier) is also advantageous in connection with connecting a number of cell culture chambers/additional cell culture chambers (30, 80) in series as they do not block for connection in the first/length direction.

FIG. 5a shows the cell culture chamber 30 including circumferential access port 74 and planar access port 76. These ports provide access to the cell culture of the cell culture chamber 30 and may be provided with a sensor for monitoring the conditions of the cell culture, for instance the glucose level. Locking flange(s) 58 engaging with corresponding flange(s) 56 (FIG. 2b) or 78 (FIG. 5b) is also provided.

FIG. 5b shows an additional cell culture chamber 80 that can be inserted between the first cell culture chamber 30 and the double walled vessel containing the spent and fresh growth media. The additional cell culture chamber 80 contains an orifice or hole 82 for allowing media, e.g. liquid media, to flow between the two cell culture chambers 30 and 80. Accordingly, these cell culture chambers may be regarded as being arranged in series. A conduit 84 is also arranged, which allows liquid flow between fresh media chamber 14, through conduit 22 and via (without mixing) at least one additional cell culture chamber 80 to the cell culture chamber 30. Thus, further additional cell culture chambers may be arranged therein. For a connected series of cell culture chambers it is noted, that the cell culture chamber at one or both ends (i.e. the first and/or the last) may comprise a double vent (see e.g. 140 elsewhere) located on the front or back rather than a top-side port 74, while any cell culture chambers in-between should comprise a top-side port 74 or any other suitable port.

FIG. 6 is an embodiment of the bioreactor 10″ according to the second aspect as disclosed herein, where the cell culture chamber 30 is an annulus of the vessel 12. Hence, the annular compartment 22, 22′ is the cell culture chamber 30 and which is in fluid communication with said spent media chamber 16 and said fresh media chamber 14. A more compact construction results. The vessel 12 is preferably provided with an arrangement 86 for opening and closing the bioreactor. Access ports, sensor locations and humidification systems have been omitted for clarity but can of course be included in a similar manner as that described above. Such embodiments may also be combined with a circumferential gas exchanger (and e.g. circumferential humidifier) according to the fifth aspect and as disclosed herein.

FIG. 7 illustrates a bioreactor system 88 according to the third aspect as disclosed herein. The system may be mounted on a platform 90 and comprises the bioreactor 10 as disclosed herein comprising cell culture chamber 30 at a first flat end of the double wall vessel 12. Instead of the bioreactor embodiment 10 exemplified in FIG. 1-3, the bioreactor embodiment 10′ exemplified in FIG. 4, the bioreactor embodiment 10″ exemplified in FIG. 6, and the bioreactor embodiment 100 exemplified in FIGS. 8-11 may also be used. Retaining rollers 92 and 94 or the like are provided respectively underneath and on top of the vessel 12 to support and/or enable rotation of the bioreactor 10. A drive element in the form of a drive wheel 96 are affixed the vessel 12 and thus the bioreactor 10, thereby providing the rotation. This function may also be carried out by 92 and/or 94. The system comprises also a retaining block 98 for supporting the piston 40 which includes piston shaft 42 and movable wall 38, and a drive unit 500 to move the retaining block 98. An optional conduit 502, such as a tube, is provided, which runs through the central axis of piston e.g. inside the piston shaft 42 to the outside 504. Direct and simple sampling of the cells from the cell culture chamber 30 is thus obtained.

FIG. 8 schematically illustrates an alternative embodiment of a vessel of a bioreactor according to the first aspect. Illustrated is an embodiment of a vessel 12 of a bioreactor (see e.g. 10, 10′, 10″, 100 in other Figs.) as disclosed herein corresponding to the vessel of FIG. 1 except as explained in the following. For clarity, not all elements are shown again in FIG. 8. FIG. 8A illustrates a first end view of the vessel 12, FIG. 8B illustrates a perspective side/top view of the vessel 12, and FIG. 8C illustrates a lengthwise cross-sectional view of the vessel 12.

Like in FIG. 1 and as disclosed herein, the vessel comprises a first end 24, a second end 26, a spent media chamber 16, a fresh media chamber 14, and a movable wall 38 connected to a piston shaft 42 of a piston 40 and separating the spent 16 and the fresh media chamber 14 as explained and disclosed herein.

The vessel in FIGS. 8A-8C is shown without a cell culture chamber but may e.g. be used with one such as a cell culture chamber of FIG. 1 (see e.g. 30 in FIG. 1) and in particular with a removable end (see e.g. 46 in FIGS. 1 and 3) comprising a cell culture chamber as disclosed herein and e.g. as illustrated in FIG. 8D.

As illustrated in FIG. 8C, the vessel 12 rather than comprising an outer and inner wall (see e.g. 18 and 20 in FIG. 1) defining an annular compartment (or sub-compartment(s)), the vessel 12 (of the shown and corresponding embodiments) comprises a least one single wall 18′ (i.e. not a double-wall) and at least one media conduit 22′ replacing the annular compartment or sub-compartment(s). In some embodiments where the vessel is generally cylindrical, the vessel 12 comprises a single generally cylindrical wall 18′.

Each of the at least one media conduit 22′ is in fluid connection with the fresh media chamber 14 via a respective orifice or valve 36′ through the (single) wall 18′ allowing (liquid) media to flow from the fresh media chamber 14 to the respective media conduit 22′ upon proper movement of the movable wall 38. Each media conduit 22′ further comprises (typically arranged at an opposite or other end than the orifice 36′) an additional orifice or valve 32 aligning, corresponding, or being coincident with an inlet orifice or valve (see e.g. 32 in FIG. 1) of the cell culture chamber as described in connection with FIG. 1 (but where the dimension(s) of the cell culture chamber may be a bit different than in FIG. 1 to align with the media conduit(s) 22′ rather than an annular compartment or sub-compartment(s). The first end 24 of the vessel 12 further comprises an orifice 34, or alternatively a valve or similar, for allowing (liquid) media from the culture chamber to enter into the spent media chamber 16 where the orifice or valve 34 aligns, corresponds, or is coincident with an outlet orifice or valve (see e.g. 34 in FIG. 1) of the cell culture chamber.

In some embodiments and as shown in particular in FIG. 8A, the (at least one) media conduit 22′ is (are) arranged adjacent to the wall 18′ outside the main part of the vessel 12 on or adjacent to an outer surface of the wall 18′. In alternative embodiments, the (at least one) media conduit 22′ is (are) arranged adjacent to the wall 18′ inside the main part of the vessel 12 on or adjacent to an inner surface of the wall 18′. In principle, there could also be a mix of inner and outer media conduits 22′.

The vessel 12 of FIG. 8 is illustrated to comprise only a single media conduit 22′ but in other embodiments, the vessel 12 may comprise a plurality media conduits 22′. In embodiments, with a plurality of media conduits 22′, they may e.g. be located equidistantly (inside and/or outside) about the (circular) perimeter of the single wall 18′.

FIG. 8D illustrates the vessel 12 with a removable lid or the like (as in FIG. 1) comprising the cell culture chamber 30 attached at one end with an orifice or valve 32 aligning with a media conduit 22′.

FIG. 9 schematically illustrates a front view (shown to the right in the Figure) and a cross sectional side view (shown to the left in the Figure) of embodiments of a bioreactor 100 according to some embodiments and as disclosed herein comprising a circumferential gas exchanger and a circumferential humidifier according to the fifth aspect. In the following and herein, a ‘bioreactor’ is equally referred to as ‘a cell culture chamber device’.

Illustrated (see both views) is a cell culture chamber device 100 as disclosed herein. The cell culture chamber device 100 comprises an enclosure 30 (also in the following and herein referred to as a cell culture chamber; see e.g. 30 also in other Figs.) as disclosed herein defined by a first end 111, a second end 112, and at least one connecting wall 18′ connecting the ends 111, 112. The enclosure 30 is e.g. comprised by a housing/a main housing 105 where the housing/main housing 105 is cylindrical (as an example) and centrally (as an example) comprises the enclosure 30. In the shown and corresponding embodiments, the at least one connecting wall 18′ is constituted by a (e.g. supported) circumferential gas permeable membrane 72′ arranged along or as a circumferential part, i.e. the perimeter or part thereof, of the enclosure 30 and being configured for exchange of gases, e.g. oxygen and carbon dioxide. The circumferential gas permeable membrane 72′ may e.g. correspond to the membrane (72′) shown and explained in connection with FIG. 4 and e.g. be a semipermeable membrane.

Humidification of the atmosphere close to or in the vicinity of the circumferential gas permeable membrane 72′ will typically reduce or avoid cell culture media evaporation and may for certain cell culture media furthermore greatly facilitate the exchange of gases through the circumferential gas permeable membrane 72′. Cells produce CO₂ which in solution combines with water to form bicarbonate (which is acidic). Humidification of the atmosphere results in the outer surface of the circumferential gas permeable membrane 72′ becoming humid and this facilitates the escape of CO₂ from the culture media and in doing so slow the acidification process. This process occurs in types of cell culture that do not rely on CO₂ to buffer the media (e.g. those that contain HEPES, a zwitterionic sulfonic acid buffering agent). The most widely used types of growth media rely on bicarbonate in the media and CO₂ in the atmosphere to buffer the pH of the media. Here also humidification of the outer surface of the circumferential gas permeable membrane facilitates the ‘capture’ or ‘release’ of CO₂ improving stabilisation of the pH of the medium. Humidification can be provided by the cell culture chamber device 100 being located in a humidified incubator or by a humidifier as described in the following.

The cell culture chamber device 100 comprises, as shown by the front view (to the right in the Figure), a gas exchange intake and outlet for a gas exchanger of the cell culture chamber device 100 that may be any suitable intake, conduit, etc. Preferably, and as shown, the gas exchange intake and outlet is in the form of a double vent or similar 140 connecting the circumferential gas exchanger with outside or ambient air or gas of the cell culture chamber device 100. Alternatively, the gas exchange intake and outlet is like 70′ in FIG. 4. In the shown embodiment, the double vent 140 is, as an example, located on a front side or front facing side (see also later Figures) or similar of a housing or main housing 105. In this particular (and corresponding embodiments), the gas exchanger comprises a gas permeable membrane 72′ configured to exchange gases, e.g. oxygen and carbon dioxide, with the enclosure 30/the content of the enclosure (e.g. cell culture media). In particular, oxygen may be provided into the enclosure 30 and carbon dioxide may be removed from the enclosure 30. In the shown and corresponding embodiments, the membrane 72′ constitutes the (at least one) connecting wall 18′ of the enclosure 30 or one or more parts thereof.

The gas exchange intake and outlet/the double vent 140 is in fluid connection with the membrane 72′ thereby connecting the membrane 72′ with outside or ambient air or gas of the cell culture chamber device 100. In at least some embodiments, the double vent 140 is configured to operate according to the Coand\check{a} effect or principle. In such embodiments, a wall or other suitable barrier 151 (indicated in the Figure by a straight dashed line) is located in-between the two respective vents of the double vent 140 separating and sealing them from each other at this location, i.e. in this particular example separating and sealing them in the shortest direction between them. However, the two vents of the double vent 140 are in fluid connection with each other via another path inside the housing 105 of the cell culture chamber device 100 and are also in fluid connection with at least parts of the gas exchange membrane 72′ e.g. via one or more conduits, open spaces, cavities, etc. When the cell culture chamber device 100 is rotated anticlockwise, ambient air or gas is sucked into and out of the cell culture chamber device 100 via the double vent 140 as indicated by the arrows of the front view and cross-sectional side view of FIG. 9. As can be seen, air or gas is, during anticlockwise rotation, more specifically sucked into the cell culture chamber device 100 by the left (in the front view) vent 140 as indicated by the arrow going from black to grey and expelled outside the cell culture chamber device 100 by the right (in the front view) vent 140 as indicated by the arrow going from grey to black creating an internal air flow 310 with a direction as indicated by the light grey dashed circular arrow. This is the case for anticlockwise rotation. If the cell culture chamber device 100 is rotated clockwise, the direction of the airflow 310 inside the housing 105 will reverse due to symmetry, i.e. the light grey dashed circular arrow will be clockwise and air or gas is sucked in by the right vent 140 and expelled by the left vent 140.

In this way, an effective air flow 310 is readily provided being in contact with the membrane 72′ and the ambient gas or air of the cell culture chamber device 100 thereby e.g. expediently adding oxygen and removing carbon dioxide to/from the membrane 72′ and thereby to/from the content of the enclosure 30.

In some further embodiments, the degree of air movement or flow 310 can be regulated by regulating the respective sizes of openings of the vents of the double vent 140 for example with a slider or small or differently sized plugs or in another suitable manner.

In some further embodiments (and as shown), the cell culture chamber device 100 optionally further comprises a circumferential humidifier or humidification or moisturising element or system (herein equally referred to as humidifier) 62′ configured to humidify or moisturise air or gas at least in the vicinity of the membrane 72′ (at least parts thereof). A humidifier will greatly enhance a gas exchange between the content of the enclosure 30 and the ambient air or gas and will furthermore reduce or eliminate water or liquid loss from the enclosure 30 when containing a liquid or aqueous solution. The effect is so significant that the cell culture chamber device 100 will normally be able to be used in an incubator without additional humidification. This is advantageous since it typically will reduce a risk of infection in the incubator and also enables simplification of the incubator.

In some such embodiments, the circumferential humidifier 62′ comprises (or is connected to) one or more liquid or moisturising reservoirs or elements. It is advantageous if the weight distribution of the circumferential humidifier 62′ is at least somewhat uniformly distributed, at least to some extent, about a central axis or a rotational axis of the cell culture chamber device 100. It is also an advantage if such one or more liquid or moisturising reservoirs or elements has, or provides, a relatively large surface area for evaporation.

There are several expedient possibilities for humidifying or moisturising air or gas at least in the vicinity of the membrane 72′ (at least parts thereof).

In some embodiments, the circumferential humidifier 62′ comprises an element or reservoir (see e.g. 62, 62′ in FIGS. 3, 4) containing (preferably sterile) liquid water or other moisturising liquid e.g. with one or more suitable filters, outlets, further (gas permeable and particularly semipermeable) membranes (see e.g. 66′ in FIG. 4), etc. interfacing the water or liquid with the air flow 310 thereby humidifying or moisturising the air flow 310. The element or reservoir may e.g. be a single circumferential unit or alternatively be several separate and distinct units (e.g. evenly distributed about the central and/or rotational axis).

In alternative embodiments, the circumferential humidifier 62′ comprises one or more of a water or solute-containing material such as a gel, sponge, a particulate material (e.g. water-beads, aqua-beads, slush powder, or water gel powder, etc.) that readily provides evaporation of water or liquid and efficiently influences the air flow 310. Such solid humidifying or moisturising elements may be supported or secured in the housing 105 e.g. by or to an (open) enclosure, a wall or other support structure (e.g. 145 in the following Figures).

In case of water-beads or a gel, these may be secured, adhered, pasted, etc. to an inner wall (as mentioned e.g. or preferably uniformly about the central and/or rotational axis) of the main housing/housing 105, whereby support structures are not necessary.

For embodiments not comprising a water or liquid reservoir (e.g. water-beads, gel, etc. as mentioned above), it is possible to locate such directly in a conduit, cavity, open space, etc., comprising the air flow 310, thereby greatly increasing the humidifying or moisturising effect of the air flow 310 and enabling reduction of overall space/foot-print of the cell culture chamber device 100. This avoids the need for a separate reservoir such as 62′ in FIGS. 4 and 9.

It is noted, that for embodiments without a humidifier (e.g. for use in a humidified incubator or other), the shown cell culture chamber device 100 will not comprise the illustrated circumferential humidifier 62′ and may have a reduced size as a result.

FIGS. 10A-10E respectively schematically illustrates a front, a first (‘right’) side view, a first cross sectional view (AA), a second cross sectional view (CC), and a third cross sectional view (BB) of one exemplary embodiment of a cell culture chamber device as disclosed herein.

Illustrated in FIG. 10A is a front view of an exemplary preferred embodiment of a cell culture chamber device as disclosed herein. Illustrated is a front of a cell culture chamber device 100. In this particular (and corresponding embodiments) a floor, bottom, or wall of a cover 102 (see e.g. also 102 in FIG. 11) constitutes a first end 111 (or a part or window 113 thereof) of the cell culture chamber device 100. The floor or bottom of the cover 102 form an enclosure (see e.g. 30 in FIGS. 1, 3, 5A and 7 and 30 in FIG. 9) as disclosed herein together with a first or central housing 101 as will be more apparent from some of the following figures. In some embodiments (and as shown in FIGS. 100, 10D, 10E, 11, etc.), the first or central housing 101 comprises a central cavity for (e.g. or preferably releasable) receipt of at least a part of the cover 102 and more particularly (in the shown and corresponding embodiments) for receipt of the floor or bottom of a cover 102. The first or central housing 101 and the cover 102 may e.g. comprise respective releasable securing elements (such as snap fit, bayonet, friction fit, etc. elements) to releasably secure them together. Alternatively, they may be fixed non-releasably to one another or e.g. be integrally formed.

The cell culture chamber device 100 of FIGS. 10A-10E is, as an example, shaped substantially cylindrically with a circular first end.

In this particular (and corresponding embodiments), the central housing 101 additionally comprises a gas exchange circuit, element, or system in the form of a circumferential gas exchange system comprising a circumferential gas permeable membrane (not shown; see e.g. 72′ in FIGS. 4 and 62′ and 72′ in FIGS. 9, 10D, and 11). As mentioned, the floor or bottom of the cover 102 constitutes a first end 111 (or a part or window 113 thereof) of the cell culture chamber device 100.

In this particular (and corresponding embodiments), the central housing 101 furthermore comprises a circumferential humidifier (not shown) as disclosed herein and e.g. as explained in connection with FIG. 4 or 9). Some alternative embodiments of the cell culture chamber device 100 do not comprise any humidifier, e.g. for use in a humidified incubator or other.

The central housing 101 optionally comprises a gas exchange intake and outlet for a gas exchanger as disclosed herein (see e.g. 130, 140, 151, 310, etc. in FIGS. 9, 10, and 11) in the form of a double vent 140 located on the front of the central housing 101. The double vent 140 has been described in more detail e.g. in connection with FIG. 9.

Further indicated are three cross-sections designated AA (shown in FIG. 10C), BB (shown in FIG. 10E), and CC (shown in FIG. 10D).

In some embodiments (and as shown), the cell culture chamber device 100 further comprises a closable and/or sealable (first) port 76 providing access for a user to the inside of the enclosure e.g. for taking out a sample from the enclosure (e.g. removing spheroids), emptying or filling the enclosure, etc. In the shown embodiment, the closable and/or sealable port 76 comprises a conduit (from the inside of the enclosure to outside the cell culture chamber device 100) and e.g. a simple plug or similar 160. The port may (alternatively or in addition) advantageously be located on the top of the cell culture chamber device 100 as this may avoid or reduce bubble formation, e.g. by allowing for overflow. Such a ‘top-side’ port is e.g. shown as 74 in FIGS. 1, 3, 10, and 11.

In some embodiments (and as shown), the cell culture chamber device 100 further comprises one or more fiducial and/or identification markers, here an identification code 155 and a fiducial marker 180. The identification code 155 is preferably unique to the particular cell culture chamber device 100. The fiducial marker 180 enables determination of the orientation of the cell culture chamber device 100. The fiducial and/or identification markers 155, 180 is/are preferably machine readable, e.g. by a suitable imaging or vision unit or system. In some embodiments, the cell culture chamber device further comprises one or more aligning elements (e.g. location bar and slit or slot, etc.) for aligning different parts (ensuring or facilitating that a part may only be received with a proper orientation by another part) of the cell culture chamber (e.g. appropriately aligning the cover 102 with the first or central housing 101). The fiducial marker 180 may e.g. be such an aligning element.

Accordingly, a very compact (lengthwise) cell culture chamber device 100 is provided, in particular because of the circumferential gas exchange system and (if present) the circumferential humidifier.

Optionally, the cover 102 comprises a number of level or fill-rate indicators 190 readily indicating an actual volume of cell culture media contained in the enclosure.

In some embodiments and as shown, the cell culture chamber device 100 further comprises one or more (here two) feet, standing elements or the like 501 enabling the cell culture chamber device 100 to stand and from rolling. This may make use of ports, inlets, etc. easier or more reliable (see e.g. port 74 herein).

Illustrated in FIG. 10B is a side view of the cell culture chamber device 100 of FIG. 10A seen from the side and from right to left (according to the orientation of FIG. 10A). The shown cell culture chamber device 100 comprises a main housing 105 receiving (e.g. permanently or in a fixed way) the central housing 101 in turn receiving (e.g. releasably) the cover 102. Further shown is a (second) port 74 (or rather a plug or valve thereof 170) that is in fluid connection with and provides (additional) access to the enclosure.

The ratio between a first extent/length (in the left right direction of FIG. 10B) and the second extent/height or diameter (in the up down direction of FIG. 10B) is about 1 to about 3-4 e.g. about 1 to about 3.5 but may be different, e.g. as disclosed herein, for other embodiments.

Illustrated in FIG. 10C is a first cross sectional view as given by A-A of FIG. 10A. Illustrated is the enclosure 30 defined by the first end 111 (being the floor or bottom of the cover 102, the second end 112 being a floor or bottom of the central housing 101, and sidewalls of the cavity of the central housing 101. Further illustrated is the main housing 105 receiving the central housing 101 and the cover 102 in a very compact way.

As mentioned, the second port 74 provides access (in addition to the first port 76) to the enclosure 30. As explained in connection with e.g. FIG. 9, the double vent 140 connects the outside or ambient air or gas of the cell culture chamber device 100 with the circumferential gas exchange system (see e.g. 130, 140, 310, etc. in FIGS. 9, 10, and 11).

As can be seen, the closable and/or sealable first port 76 and its conduit connects the inside of the enclosure 30 to outside the cell culture chamber device 100. The port walls are a part of the cover 102, allowing for easy access to the content of the enclosure 30. In a similar manner, access to the inside of the enclosure 30 is afforded via the second port 74 (with plug 170). The plug walls of 74/170 are a part of the central housing 101. It is noted, that the first port 76 and the second part 74 are arranged at different sides of the cell culture chamber device 100 enabling easy access to the enclosure from several different sides of the cell culture chamber device 100.

Further illustrated is the gas exchange intake and outlet in the form of a double vent 140 as previously explained.

The view of FIG. 100 is a central vertical cut viewed from left to right (in the orientation according to FIG. 10A).

Illustrated in FIG. 10D is a second cross sectional view as given by C-C of FIG. 10A and viewed from right to left.

Again, the enclosure 30, the first transparent end 111, the transparent or translucent second 112, the central housing 101, the cover 102, the closable and/or sealable ports 76 and 74, and the main housing 105 are illustrated.

Further shown, is the gas permeable membrane 72′ of the circumferential gas exchange system and a (part of a) grid like structure 130 of the circumferential humidifier.

Also illustrated is a wall structure element or similar 145 for holding and/or supporting a water, liquid, or moisturizing element (explained further in connection with FIG. 9) according to some embodiments of a circumferential humidifier.

In some embodiments, the cell culture chamber device 100 optionally further comprises one or more markings 115—herein as an example in the form of a number of concentric circles 115 that may give a user some fixed marks against which to see the gentle movement of the contained spheroids. The markings 115 are (as an example) arranged on the ‘outside’ of the second end 112.

The view of FIG. 10D is a vertical cut shifted off-centre to the left and viewed from right to left (in the orientation according to FIG. 10A).

Illustrated in FIG. 10E is a third cross sectional view as given by B-B of FIG. 10A.

Illustrated is the enclosure 30, the first end 111, the second 112, the cover 102, the closable and/or sealable port 76, and two wall structure elements or similar 145 for holding and/or supporting a water, liquid, or moisturizing element according to some embodiments.

The view of FIG. 10E is a horizontal central cut viewed from top to bottom (in the orientation according to FIG. 10A).

FIG. 11 schematically illustrates a perspective exploded view of the exemplary embodiment of a cell culture chamber device of FIGS. 10A-10E.

Illustrated are the elements of FIGS. 10A-10E shown in an exploded view.

FIG. 11 more clearly shows the grid like structure 130 of the circumferential humidifier and the gas permeable membrane 72′. In an assembled state of the cell culture chamber device 100, the gas permeable membrane 72′ is located adjacent to and on an inner side of the grid like structure 130 and the first end 111 is opposite the second 112 and the second end 112 is aligned with an opening 185 of the main housing 105 readily enabling inspection of the content of the enclosure from that side also if the second end 112 is transparent. Further shown is a further port 150 that aligns with the first port 74 in the assembled state.

In at least some embodiments, the material of the main housing 105, the central housing 101 (and thereby the second end 112), the cover 102 (and thereby the first end 111) may e.g. be the same material e.g. as disclosed herein elsewhere.

The embodiments of a cell culture chamber device 100 as illustrated in FIGS. 10 and 11 provides a very compact (in particular in a lengthwise direction) self-contained and fully functioning cell culture chamber device 100 or bioreactor where the gas exchanger (and if included, the humidifier) is arranged away from a central axis and/or an axis of rotation. In addition, the cell culture chamber device 100 has a petri-dish like design enabling easy and familiar handling.

1. A bioreactor (10, 10′, 100) adapted for rotation, the bioreactor comprising:
a vessel (12) comprising:
a first end (24) and a second end (26) which define a central axis (28) of the vessel (12) extending along a first direction, e.g. a length direction, of the vessel (12) from said first (24) to said second end (26), at least one wall (18, 18′, 20) running along the first direction of the vessel (12), at least one media conduit (22, 22′) defining a volume for receiving fresh or spent media;
an inner chamber defined by at least a part of a space confined within said at least one wall (18, 18′, 20) and comprising a fresh media chamber (14) and a spent media chamber (16);
a cell culture chamber (30) in fluid communication with said at least one media conduit (22, 22′) and said fresh (14) and/or spent media chamber (16); and
a movable wall (38) configured, within said inner chamber, to separate said fresh media chamber (14) from said spent media chamber (16) within said inner chamber.

2. The bioreactor (10, 10′, 100) according to claim 1, wherein
the at least one wall (18, 18′, 20) comprises an inner wall (18) and an outer wall (20), the inner (18) and outer wall (20) defining an annular compartment (22) in between said walls (18, 20),
the at least one media conduit (22, 22′) is the annular compartment (22), and
the inner chamber is defined by at least a part of, e.g. all of, the space confined within said inner wall (18).

3. The bioreactor (10, 10′, 100) according to claim 1, wherein
the at least one wall (18, 18′, 20) is a single wall (18′), and
the at least one media conduit (22, 22′) is at least one conduit (22′) arranged inside or outside the single wall (18′).

4. The bioreactor (10, 10′, 100) according to claim any one of claims 1-3, wherein said cell culture chamber (30) is arranged separately at said first end (24) of the vessel (12) and is provided with an inlet orifice (32) for allowing media from said at least one media conduit (22, 22′), e.g. said annular compartment (22) or said at least one conduit (22′), to enter into the cell culture chamber (30), and an outlet orifice or valve (34) for allowing media from said culture chamber (30) to enter into said spent media chamber (16).

5. The bioreactor (10, 10′, 100) according to any one of claims 1-4, wherein said at least one media conduit (22, 22′), e.g. said annular compartment (22) or said at least one conduit (22′), is provided with an inlet orifice (36, 36′) for allowing media from said fresh media chamber (14) to enter into the at least one media conduit (22, 22′), and an outlet orifice (32) for allowing media from the at least one media conduit (22, 22′) to enter into the cell culture chamber (30), and wherein said outlet orifice of the annular compartment corresponds to said inlet orifice of the cell culture chamber.

6. The bioreactor (10, 10′, 100) according to any one of claims 1-5, wherein at least part of said at least one wall (18, 18′, 20) is detachable, preferably as a removable end (46) of the at least one wall (18, 18′, 20) located at said first end (24) of the vessel (12), for providing access to the cell culture chamber (30).

7. The bioreactor (10, 10′, 100) according to any one of claims 1-6, wherein at least part of said at least one wall (18, 18′, 20) is transparent glass or plastic configured to permit observation of the media, cells and spheroids contained therein.

8. The bioreactor (10, 10′, 100) according to any of claims 2 and 3-7 as dependent on claim 2, wherein said inner wall (18) has a plurality of raised ridges (48) extending along the first direction of the vessel (12), and extending vertically outwards until contacting said outer wall (20), and wherein a space between said raised ridges (48) define one or more sub-compartments within said annular compartment (22).

9. The bioreactor (10, 10′, 100) according to any of claims 1-8, wherein the vessel (12) has a cylindrical or generally cylindrical shape and is adapted for rotation around a rotational, e.g. horizontal, axis (28) by one or more associated rotation elements, e.g. a drive unit, said rotational axis (28) being said central axis (28) running along the first direction of the vessel (12).

10. The bioreactor (10, 10′, 100) according to any of claims 1-9, wherein said movable wall (38) is connected to a displacement element (40) for displacing the movable wall (38) axially along the first direction of the vessel (12).

11. The bioreactor (10, 10′, 100) according to claim 10, wherein said displacement element (40) is a piston (40), said movable wall (38) being connected to said piston (40) through a piston shaft (42) which is coincident with said central axis (28) running along the first direction of the vessel (12).

12. The bioreactor (10, 10′, 100) according to claim 11, wherein a conduit (502) is provided which runs from the cell culture chamber (30) through a centre of said piston shaft (42) to at least the outside (504) of the vessel (12).

13. The bioreactor (10, 100) according to any of claims 1-12, wherein the vessel (12) or part of the vessel is constructed of a gas permeable plastic,
or
the vessel (12) includes a gas permeable membrane (72, 72′) for the exchange of gasses, such as oxygen and carbon dioxide, said gas permeable membrane (72′) being arranged along the circumferential part of the cell culture chamber (30).

14. The bioreactor (10, 10′, 100) according to claim 13, wherein a humidification system (62, 62′, 66, 66′, 68, 68′, 72, 72′) is provided between the culture chamber (30) and the external atmosphere, said humidification system comprising a liquid reservoir (62, 62′), preferably containing sterile water, an evaporation chamber (68, 68′), such as an evaporation labyrinth (68, 68′), and a filter (66, 66′, 72, 72′).

15. The bioreactor (10, 10′, 100) according to claim 14, wherein said filter (72, 72′) is said gas permeable membrane (72′) being arranged along the circumferential part of the cell culture chamber (30).

16. The bioreactor (10, 10′, 100) according to claim 14 or 15, wherein said humidification system further comprises an additional filter (66′) arranged in fluid communication with said liquid reservoir (62, 62′) and said evaporation chamber (68, 68′).

17. The bioreactor (10, 10′, 100) according to claim 16, wherein said additional filter (66′) is arranged along the circumferential part of the vessel (12) and in between said liquid reservoir (62, 62′) and said evaporation chamber (68, 68′).

18. The bioreactor (10, 10′, 100) according to any of claims 1-17, wherein the cell culture chamber (30) includes at least one access port (74, 76) and optionally a sensor, said sensor preferably being removably mounted in said access port.

19. The bioreactor (10, 10′, 100) according to any of claims 1-18, wherein said at least one wall (18, 18′, 20) includes an access port (54) to said fresh media chamber.

20. The bioreactor (10, 10′, 100) according to any of claims 1-19, wherein an additional cell culture chamber (80) is adapted in series connection with said cell culture chamber (30), said additional cell culture chamber (80) having a conduit (84) to transfer said fresh media and having an orifice (82) for allowing the media to flow from the cell culture chamber (30) to said additional cell culture chamber (80).

21. The bioreactor (10, 10′, 100) according to any one of claims 1-20, wherein the bioreactor (10, 10′, 100) includes at least one membrane for preventing at least part of the cell culture in the cell culture chamber (30) from exiting said cell culture chamber (30).

22. The bioreactor (10, 10′, 100) according to claim 21, wherein the at least one membrane is arranged over one or more of the inlet and/or outlet orifices (32, 34, 74) of the cell culture chamber (30).

23. The bioreactor (10, 10′, 100) according to any one of claims 1-22, wherein one or more additional cell culture chambers (80) are assembled on or in the vessel (12) by inserting extra additional cell culture chambers (80), preferably essentially petri-dish shaped cell culture chambers (30, 100).

24. The bioreactor (10, 10′, 100) according to claim 23, wherein at least one sensor is mounted on or in the one or more additional cell culture chambers (80) in such a way that the contents of each chamber (30) can be monitored independently.

25. A bioreactor (10, 10′, 10″, 100) adapted for rotation, the bioreactor (10, 100) comprising:
a vessel (12) including:
a first end (24) and a second end (26) which define a central axis (28) of the vessel (12) extending along a first direction, e.g. a length direction, of the vessel (12) from said first (24) to said second end (26),
at least one wall (18, 18′, 20) running along the first direction of the vessel (12);
at least one media conduit (22, 22′) defining a volume for receiving fresh media;
an inner chamber defined by at least a part of a space confined within said at least one wall (18, 18′, 20) and comprising a fresh media chamber (14) and a spent media chamber (16);
said at least one media conduit (22, 22′) being or comprising a cell culture chamber (30) in fluid communication with said spent media chamber (16) and said fresh media chamber (14);
a movable wall (38) configured, within said inner chamber, to separate said fresh media chamber (14) from said spent media chamber (16), within said inner chamber.

26. The bioreactor (10, 10′, 10″, 100) according to claim 25, wherein
the at least one wall (18, 18′, 20) comprises an inner wall (18) and an outer wall (20), the inner (18) and outer wall (20) defining an annular compartment (22) in between said walls (18, 20),
the at least one media conduit (22, 22′) is the annular compartment (22), and
the inner chamber is defined by at least a part of, e.g. all of, the space confined within said inner wall (18).

27. The bioreactor (10, 10′, 10″, 100) according to claim 25, wherein
the at least one wall (18, 18′, 20) is a single wall (18′), and
the at least one media conduit (22, 22′) is at least one conduit (22′) arranged inside or outside the single wall (18′).

28. A bioreactor system (88) for growing a cell culture or tissue, the system (88) comprising:
a bioreactor (10, 100) adapted for rotation, said bioreactor (10, 100) comprising a vessel (12) comprising:
a first end (24) and a second end (26) which define a central axis (28) of the vessel (12) extending along a first direction, e.g. a length direction, of the vessel (12) from said first (24) to said second end (26), at least one wall (18, 18′, 20) running along the first direction of the vessel (12), at least one media conduit (22, 22′) defining a volume for receiving fresh or spent media, preferably fresh media;
an inner chamber defined by at least a part of a space confined within said at least one wall (18, 18′, 20) and comprising a fresh media chamber (14) and a spent media chamber (16);
a cell culture chamber (30) in fluid communication with said at least one media conduit (22, 22′) and said fresh (14) or spent media chamber (16), preferably said spent media chamber (16); and
a movable wall (38), configured, within said inner chamber, to separate said fresh media chamber (14) from said spent media chamber (16) within said inner chamber;
wherein said movable wall (38) is connected to a displacement element (40) in the form of a piston (40), for displacing the movable wall (38) axially along the first direction of the vessel (12);
the bioreactor system (88) further comprising:
retaining rollers (92, 94) configured to support and/or enable rotation of the bioreactor (10, 100);
a drive element (96) such as drive wheel (96) for rotating the bioreactor (10, 100);
a retaining block (98) for supporting the piston (40), said piston (40) being connected to said movable wall (38) via a piston shaft (42);
a drive unit (500) for moving said retaining block (98) and thereby displacing the piston (40).

29. A bioreactor (10′) adapted for rotation, the bioreactor (10′) comprising:
a vessel (12) including:
a first end (24) and a second end (26) which define a central axis (28) of the vessel (12) extending along a first direction, e.g. a length direction, of the vessel (12) from said first (24) to said second end (26), at least one wall (18, 18′, 20) running along the first direction of the vessel (12), at least one media conduit (22, 22′) defining a volume for receiving fresh or spent media;
an inner chamber defined by at least a part of a space confined within said at least one wall (18, 18′, 20) and comprising a fresh media chamber (14) and a spent media chamber (16);
a cell culture chamber (30) in fluid communication with said at least one media conduit (22, 22′) and said fresh (14) and/or spent media chamber (16); and
a movable wall (38) configured, within said inner chamber, to separate said fresh media chamber (14) from said spent media chamber (16) within said inner chamber,
wherein the bioreactor (10′) further comprises a humidification system (62, 62′, 66, 68, 72) comprising
one or more liquid or moisturising reservoirs or elements (62, 62′),
an evaporation chamber (68, 72), e.g. an evaporation labyrinth (68) and a filter (72), the filter (72) forming one of the walls of the cell culture chamber (30), and
and a liquid or moisturizing transport element (66), e.g. a wick (66), configured to transport liquid or moisture from the one or more liquid or moisturising reservoirs or elements (62, 62′) to the evaporation chamber (68, 72).

30. The bioreactor (10, 10′, 10″, 100) according to claim 29, wherein
the at least one wall (18, 18′, 20) comprises an inner wall (18) and an outer wall (20), the inner (18) and outer wall (20) defining an annular compartment (22) in between said walls (18, 20),
the at least one media conduit (22, 22′) is the annular compartment (22), and
the inner chamber is defined by at least a part of, e.g. all of, the space confined within said inner wall (18).

31. The bioreactor (10, 10′, 10″, 100) according to claim 29, wherein
the at least one wall (18, 18′, 20) is a single wall (18′), and
the at least one media conduit (22, 22′) is at least one conduit (22′) arranged inside or outside the single wall (18′).

32. A bioreactor (10, 10″, 100) for the growing of cell cultures and tissues, the bioreactor (10, 10″, 100) comprising
a cell culture chamber (30) configured to contain a cell culture media,
a circumferential gas exchanger (130, 140, 151, 310) arranged circumferentially about or along at least a part of the cell culture chamber (30) or about a central or lengthwise axis (28) of the bioreactor (10, 10″, 100), e.g. or preferably about a predetermined rotational axis (28) of the bioreactor (10, 10″, 100), wherein the circumferential gas exchanger (130, 140, 151, 310) comprises a cavity (310) comprising a volume connecting a gas exchange interface (72′) of the cell culture chamber (30) with ambient air or gas of the bioreactor (10, 10″, 100).

33. The bioreactor (10, 10″, 100) according to claim 32, wherein the gas exchange interface (72′) is a circumferential gas permeable membrane (72′), e.g. a semipermeable membrane, configured to exchange gases, such as oxygen and carbon dioxide, with content of the cell culture chamber (30) where the circumferential gas permeable membrane (72′) is arranged circumferentially along a circumferential part of the cell culture chamber (30).

34. The bioreactor (10, 10″, 100) according to claim 33, wherein the circumferential gas permeable membrane (72′) is a connecting wall (18′) connecting a first end (111) and a second end (112) wherein the first end (111), the second end (112), and the connecting wall (18′) at least in part defines the cell culture chamber (30).

35. The bioreactor (10, 10″, 100) according to any one of claims 32-34, wherein the gas exchange interface (72′) or the circumferential gas permeable membrane (72′) is supported by at least one support structure (130), e.g. a grid like support structure (130), comprising a number of openings configured to connect the gas exchange interface (72′) or the circumferential gas permeable membrane (72′) with air or gas of the cavity (310) of the circumferential gas exchanger (130, 140, 151, 310).

36. The bioreactor (10, 10″, 100) according to any one of claims 32-35, wherein the circumferential gas exchanger (130, 140, 151, 310) is connected with the ambient air or gas of the bioreactor (10, 10″, 100) via at least one gas or air inlet and/or outlet (140).

37. The bioreactor (10, 10″, 100) according to claim 36, wherein the bioreactor (10, 100) is configured for rotation about a predetermined rotational axis (28) and wherein at least one of the at least one gas or air inlet and/or outlet (140) is a double vent or port (140) configured to, e.g. or preferably simultaneously, draw in ambient air or gas into the cavity (310) of the circumferential gas exchanger (130, 140, 151, 310) and expel air or gas out of the cavity (310) of the circumferential gas exchanger (130, 140, 151, 310) in response to the bioreactor (10, 10″, 100) being rotated about the predetermined rotational axis (28) thereby creating an air flow (310).

38. The bioreactor (10, 10′, 10″, 100) according to any one of claims 13 and 32-37, wherein the bioreactor (10, 10′, 10″, 100) further comprises
a circumferential humidifier (62′), wherein the circumferential humidifier (62′)
is arranged circumferentially about at least a part of the cell culture chamber (30) or about a central or lengthwise axis of the bioreactor (10, 10′, 10″, 100), e.g. or preferably about a predetermined rotational axis of the bioreactor (10, 10′, 10″, 100), and
comprises or is connected to one or more liquid or moisturising reservoirs or elements (62′) configured to humidify or moisturise air or gas in at least a part of a cavity (310) of the gas exchanger (130, 140, 151, 310).

39. The bioreactor (10, 10″, 100) according to claim 38, wherein the one or more liquid or moisturising reservoirs or elements (62′) is/are configured to humidify or moisturise air or gas in vicinity of or being adjacent to at least a part of a gas exchange interface (120) or a gas permeable membrane (120).
